(19) United States
(12) Patent Application Publication
(10) Pub. No.: US 2009/0198059 A1
(43) Pub. Date: Aug. 6, 2009

(54) Process for the preparation of polymorphs, solvates of aripiprazole using aripirazole acid salts

(76) Inventors: Chava Satyanarayana, Secunderabad (IN); Gorantla Seeta Ramanjaneyulu, Secunderabad (IN); Indukuri Venkata Sunil Kumar, Hyderabad (IN); Ketavarapu Narasimha Rao, Hyderabad (IN); Koneru Sridhar, Hyderabad (IN)

Correspondence Address: LADAS & PARRY LLP, 26 WEST 61ST STREET, NEW YORK, NY, 10023, US

(21) Appl. No.: 11/665,405

(22) PCT Filed: Sep. 13, 2004
(86) PCT No.: PCT/IN04/00285

§ 371 Date: Apr. 13, 2007

(51) Int. Cl. C07D 401/12; C07D 215/227

(52) U.S. Cl. 544/363; 546/158

(57) ABSTRACT

The present invention relates to Aripiprazole, a useful agent for antipsychotic. The present invention also provides new acid addition salts of Aripiprazole and process for the preparation of polymorphs and solvates of Aripiprazole using Aripiprazole acid salts, their interconversion and the process for preparation of Aripiprazole acid salts.

The present invention relates to a process for the preparation of polymorphs, solvates of Aripiprazole using Aripiprazole acid salts, their interconversion and the process for preparation of Aripiprazole acid salts

### BACKGROUND OF THE INVENTION

U.S. Pat. No. 4,734,416 and U.S. Pat. No. 5,006,528 discloses the Aripiprazole, 7-(4-[4-(2,3-dichlorophenyl)-1-piperazinyl]butoxy)-3,4-dihydro-2(1H)-quinolinone or 7-{4-[4-(2,3-dichlorophenyl)-1-piperazinyl]butoxy}-3,4-dihydro carbostyril, is a typical antipsychotic agent useful for the treatment of Schizophrenia, having the formula as given below.

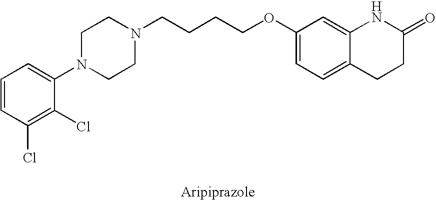

U.S. Pat. No. 5,006,528 discloses preparation of Aripiprazole and its pharmaceutically acceptable acid-addition salts. The process for the preparation of acid salts involves reaction of Aripiprazole with a pharmaceutically acceptable inorganic acids such as hydrochloric acid, sulfuric acid, phosphoric acid, hydrobromic acid and the like; organic acids such as oxalic acid, maleic acid, fumaric acid, maleic acid, tartaric acid, citric acid, benzoic acid and the like as per Scheme-1.

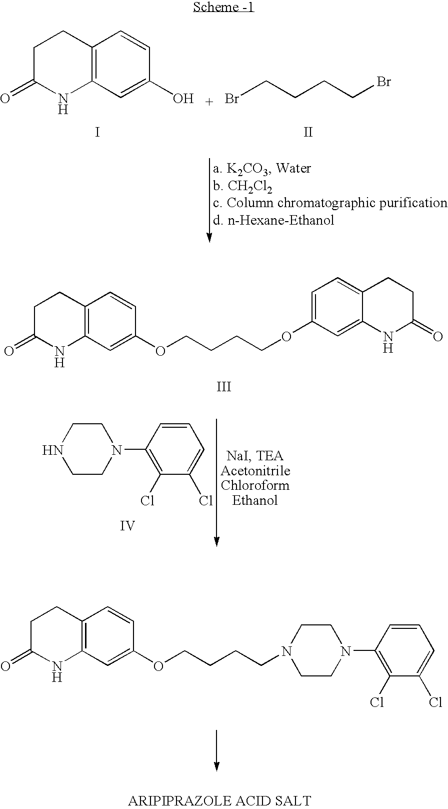

The product Aripiprazole obtained by the above process has melting point of 139.0°±139.5° C.

The process involves purification of the intermediate, 7-(4-bromobutoxy)-3,4-dihydrocarbostyril (III) by silica gel column chromatography to remove impurities formed during the reaction. The process further involves two recrystallizations of Aripiprazole from ethanol to obtain the pure Aripiprazole though compromising on yields by increasing the operational cost of the product.

PCT publication WO 03/026659 discloses low hygroscopic forms of Aripiprazole and the process for their preparation from the Aripiprazole hydrate Form ‘A’. It further states that the anhydrous Aripiprazole made by the Japanese patent publication No. 191256/1990, yields the Aripiprazole, which is significantly hygroscopic. As per PCT publication WO 03/026659 anhydrous crystals of Aripiprazole exist as type-I crystals and type-II crystals. Further discloses that the type-I crystals are prepared by recrsytallization from ethanol solution of Aripiprazole or by heating Aripiprazole hydrate at 80° C. and type-II crystals by heating type-I crystals at 130 to 140° C. for 15 hrs.

PCT application Publication WO 03/026659 discloses process for the Aripiprazole polymorphic form-B by heating the Aripiprazole hydrate ‘A’ at 90-125° C. for about 3-50 hrs. The process for Polymorphic Form-C is by heating the Aripiprazole anhydrous to a temperature of 140-150° C. The process for Form-D is recrystallization from toluene; process for Form-E is heating with acetonitrile or by recrystallization from acetonitrile and the process for Form-F is by heating the suspension of anhydrous Aripiprazole in acetone. The polymorphic Form-G is by heating to 170° C. for at least 2 weeks in a sealed tube, which is a glassy mass.

PCT publication WO 03/026659 further discloses the characterization data X-ray diffraction pattern; IR absorption and DSC of Form B, Form C, Form-D, Form-E, Form-F and Form-G. It further reported the melting point of Aripiprazole anhydrous Form B as 139.7° C.

A drawback of the disclosed process is that it produces a mixture of polymorphic forms.

### SUMMARY OF THE INVENTION

The main object of the present invention is to provide process for the preparation of pure polymorphs of Aripiprazole.

Another object of the present invention is to provide process for the preparation of Aripiprazole solvates.

Yet another object of the present invention is to provide process for preparation of polymorphs of Aripiprazole using acid salts.

Another object of the present invention is to provide process for the preparation of Aripiprazole solvates using acid salts.

Another object of the present invention is to provide process for the preparation of Aripiprazole form-B.

Another object of the invention is to provide process for the preparation of Aripiprazole form-I.

Another object of the invention is to provide process for the preparation of Aripiprazole form-D.

Another object of the invention is to provide process for the preparation of Aripiprazole form-A.

Yet another object of the invention is to provide process for the preparation of Aripiprazole methanol solvate for its use in the preparation of polymorphs of Aripiprazole.

Yet another object of the invention is to provide finger printing of Aripiprazole form-I Yet another object of the invention is to provide finger printing of Aripiprazole acid salts.

Yet another object of the invention is to provide finger printing of the Novel Aripiprazole solvates.

Thus according to the present invention Aripiprazole acid salts on treatment with base in mixture of water-organic ester solvent, followed by separation and concentration of the organic layer, then maintaining the organic layer at high temperature, cooling, followed by isolation and drying gives Aripiprazole form-B.

Aripiprazole acid salts on neutralization with base in mixture of water-water immiscible organic solvent followed by separation and concentration of the organic layer, then adding acetic acid and raising the temperature, followed by addition of ante-solvent, cooling, isolation and drying gives Aripiprazole acetic acid solvate.

Aripiprazole acid salts on neutralization with base in mixture of water and water immiscible organic solvent followed by separation and concentration of the solvent, then dissolution in organic polar solvent and raising the temperature, followed by addition of ante-solvent, cooling or optionally direct crystallization from organic polar solvent, isolation and drying gives Aripiprazole form-I.

Aripiprazole acid salts on neutralization with base in mixture of water-water immiscible organic-solvent followed by separation and concentration of the organic layer, then addition of alcohol followed by crystallization gives the Aripiprazole alcohol solvates.

Another embodiment of the present invention is process for preparation of Aripiprazole acid salts. Reaction of 7-(4-bromobutoxy)-3,4-dihydrocarbostyril (III) with 1-(2,3-dichlorophenyl)piperazine (IV) in organic polar solvent in presence of sodium iodide and triethylamine, followed by removal of solvent, dissolution of residue in mixture of water-water immiscible solvent, separation and concentration of the organic layer followed by treatment with acid results in Aripiprazole acid salts.

Alternatively Aripiprazole acid salts are prepared by reaction of 7-(4-bromobutoxy)-3,4-dihydrocarbostyril (III) with 1-(2,3-dichlorophenyl)piperazine (IV) in presence of sodium iodide and triethylamine in short chain alcohol followed by cooling results in crude Aripiprazole, which on dissolution in water immiscible solvent, treatment with acid, crystallization results in Aripiprazole acid salts.

7-(4-bromobutoxy)-3,4-dihydrocarbostyril (III) is prepared by the reaction of 7-Hydroxy-3,4-dihydrocarbostyril (I) with 1,4-dibromobutane (II) in presence of alkali hydroxide, phase transfer catalyst in alcohol, followed by removal of insolubles, distillation of solvent, excess. 1,4-dibromobutane, isolation of 7-(4-bromobutoxy)-3,4-dihydrocarbostyril (III) by addition of alcohol, cooling the resultant mass followed by isolation and washing with hydrocarbon. (Scheme-2)

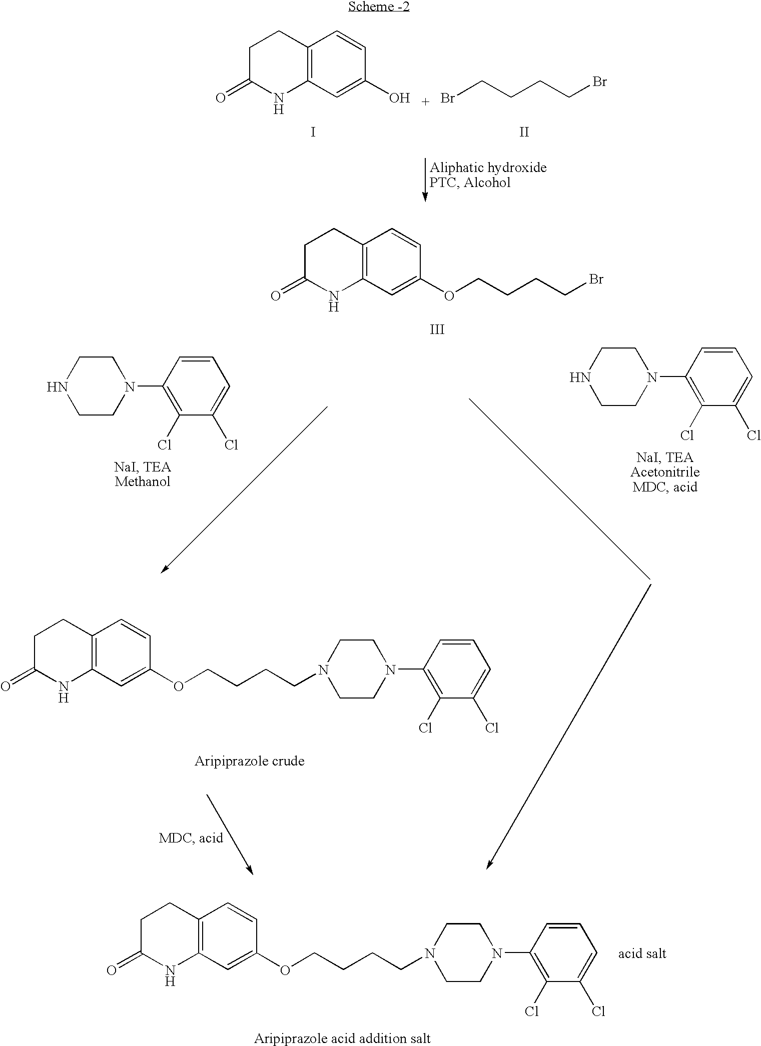

By isolating Aripiprazole as Aripiprazole acid salts minimum of 98% purity is achieved as compared to reported processes, which yield purity of 80-85%.

Yet another embodiment of the present invention is preparation of polymorphs of Aripiprazole using Aripiprazole methanol solvate. Aripiprazole methanol solvate on dissolution in organic solvent, addition of acetic acid, raising the temperature, addition of ante-solvent, cooling, followed by isolation and drying gives the Aripiprazole acetic acid solvate.

Aripiprazole methanol solvate, Aripiprazole acetic acid solvate can be converted into other crystalline forms of Aripiprazole such as Aripiprazole Form-A, Form-B, Form-D, Aripiprazole Type-I crystals and Type-II crystals by appropriate methods as per Scheme-3.

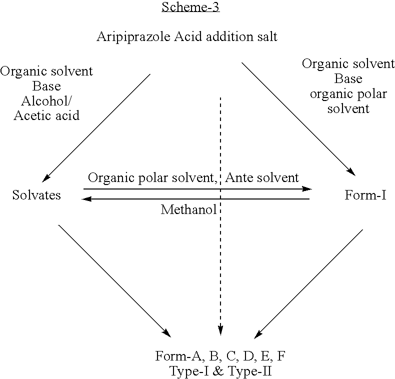

Aripiprazole acid salts used for the preparation of polymorphs of Aripiprazole in the present invention are Aripiprazole p-toluene sulfonate, benzenesulfonate, citrate, salicylate, hydrobromide and the solvates used for the preparation of Aripiprazole polymorphs are the acetic acid solvate and methanol solvate. These Aripiprazole methanol solvate, Aripiprazole acetic acid solvate, Aripiprazole form-I are to novel compounds and have characterized by chemical, IR, NMR, Mass spectral analysis and XRD.

### BRIEF DESCRIPTION OF THE DRAWINGS

FIG. 1X-ray diffraction pattern of the Aripiprazole form-B

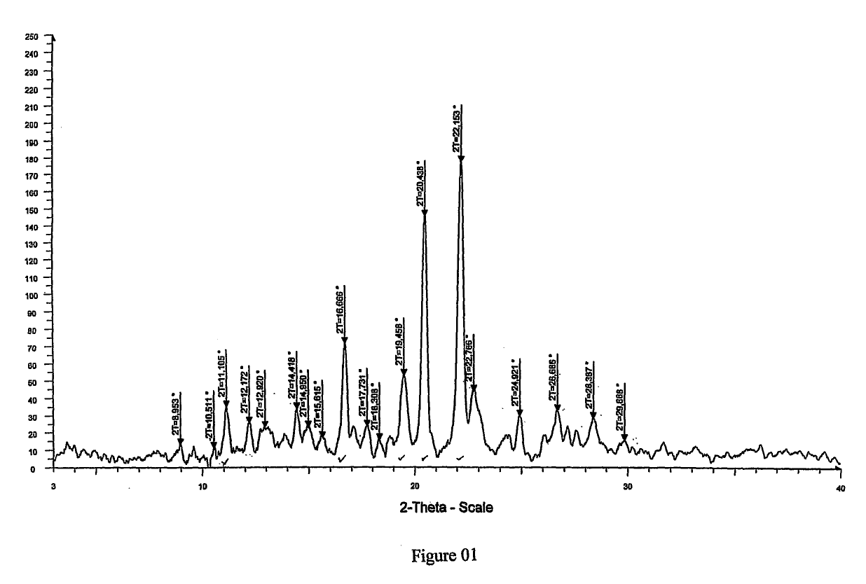

FIG. 2 FTIR spectrum of the Aripiprazole form-B

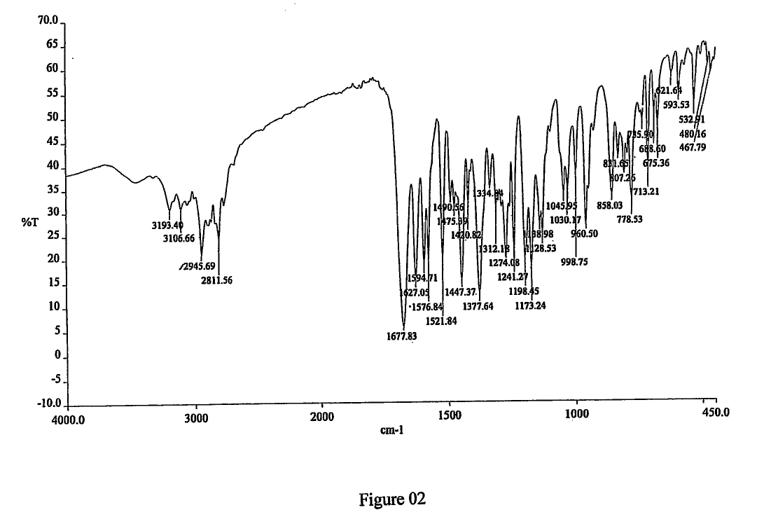

FIG. 3 DSC of the Aripiprazole of form-B

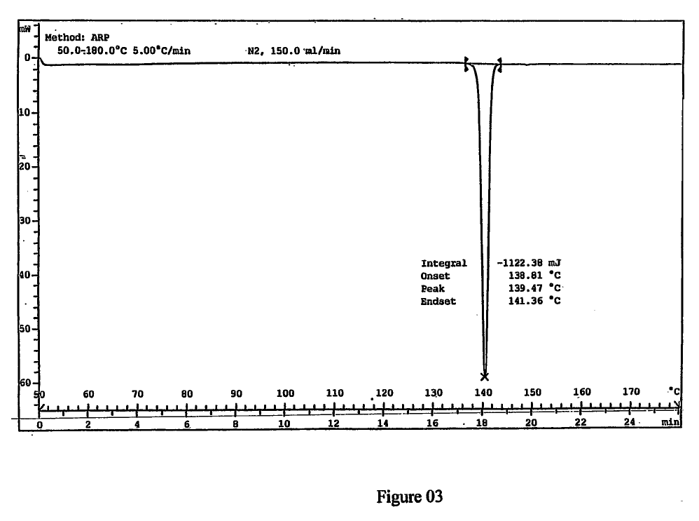

FIG. 4 X-ray diffraction pattern of the Aripiprazole form-I

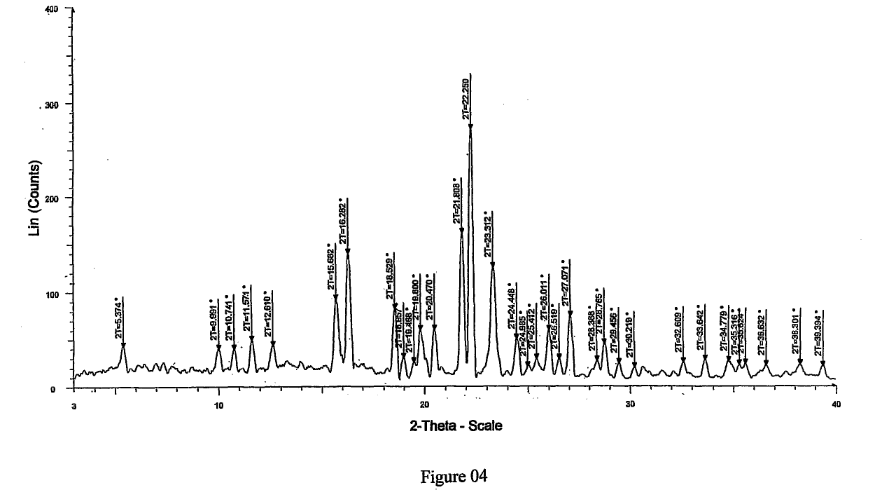

FIG. 5 FTIR spectrum of the Aripiprazole form-I

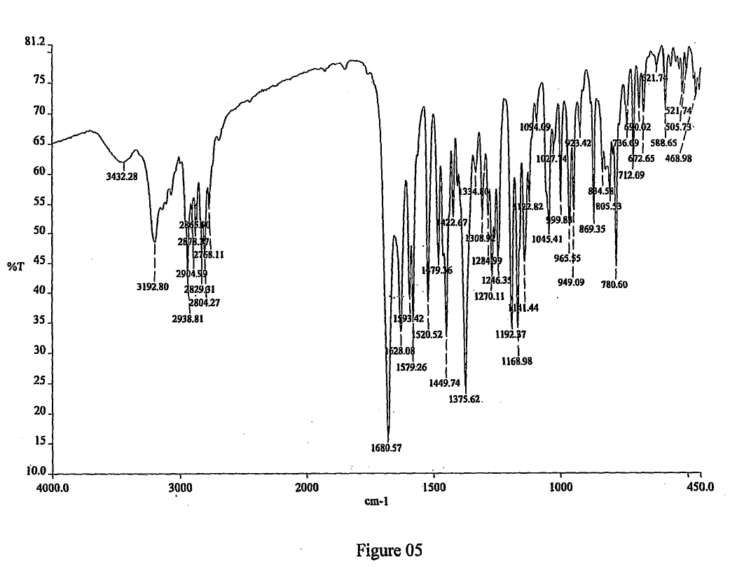

FIG. 6 DSC of the Aripiprazole of form-I

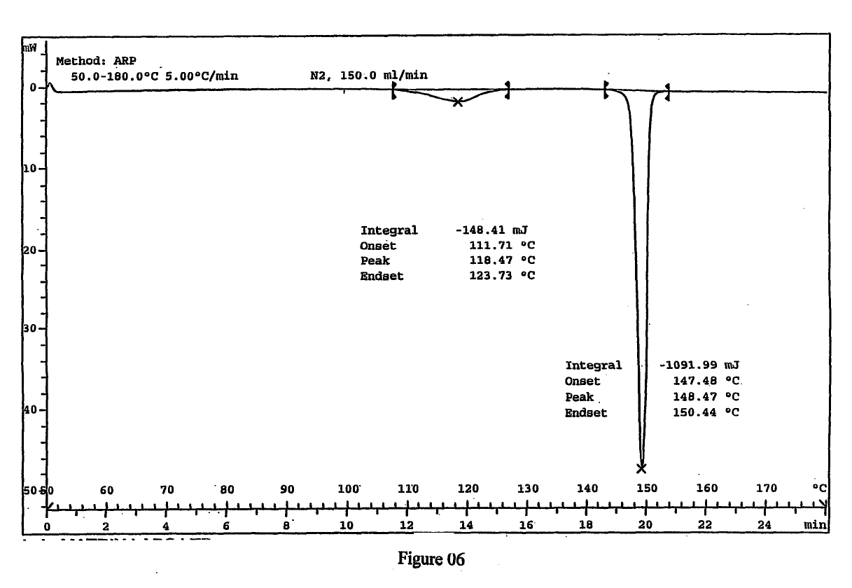

FIG. 7 XRD of the Aripiprazole methanol solvate

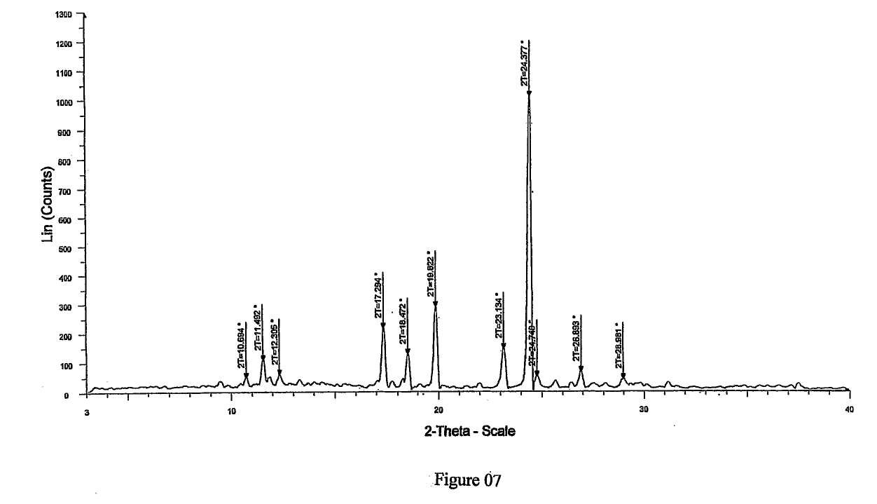

FIG. 8 FTIR of the Aripiprazole methanol solvate

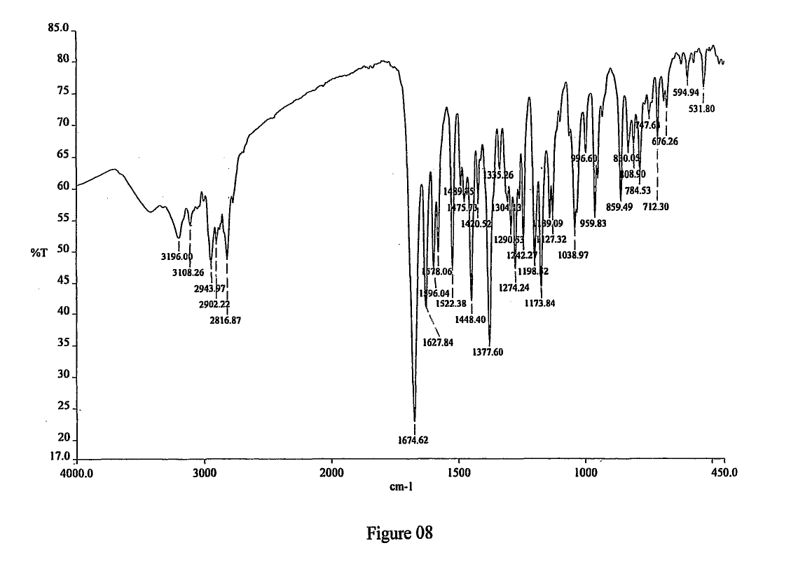

FIG. 9 TGA of the Aripiprazole methanol solvate

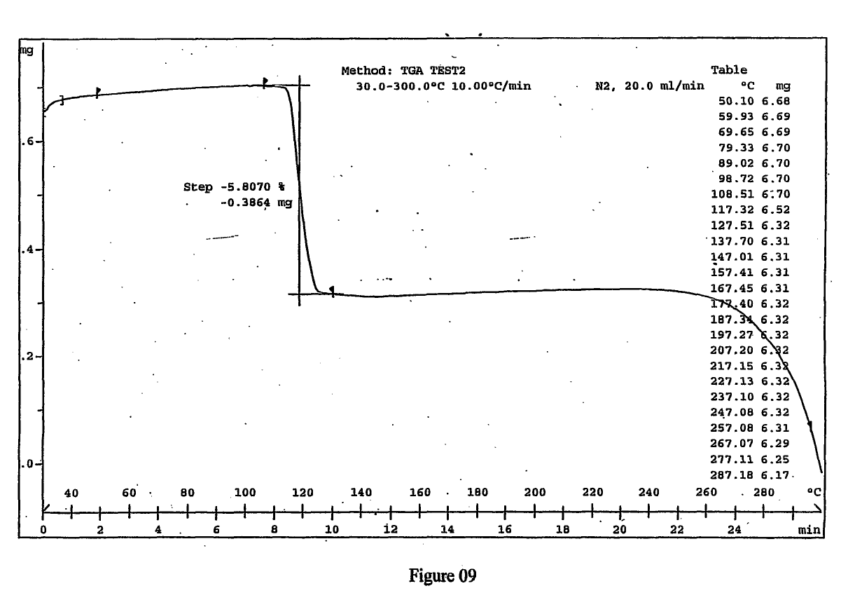

FIG. 10 XRD of the Aripiprazole acetic acid solvate

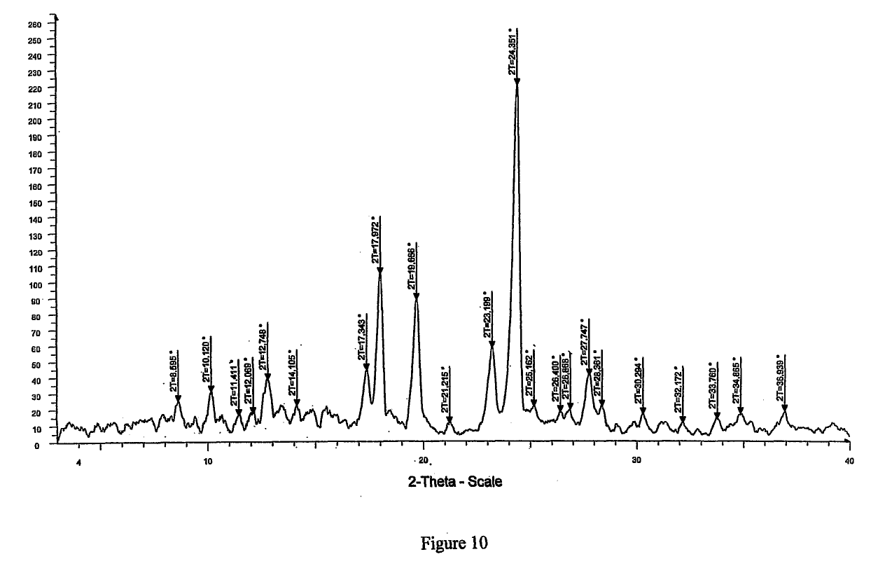

FIG. 11 FTIR of the Aripiprazole acetic acid solvate

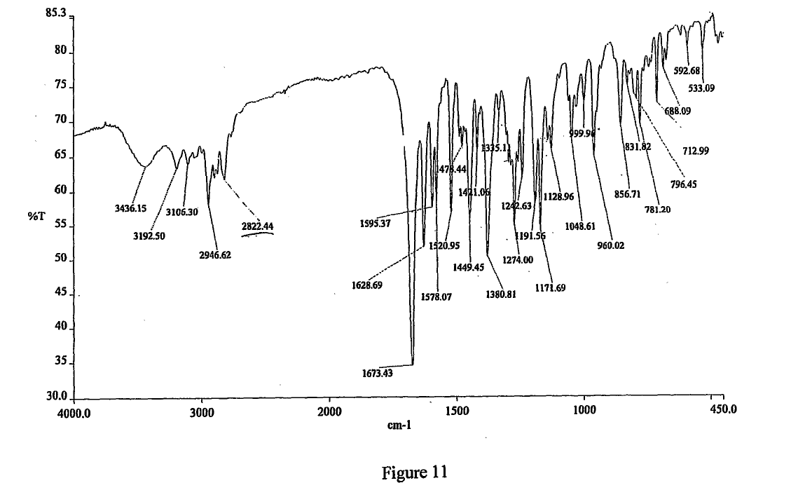

FIG. 12 TGA of the Aripiprazole acetic acid solvate

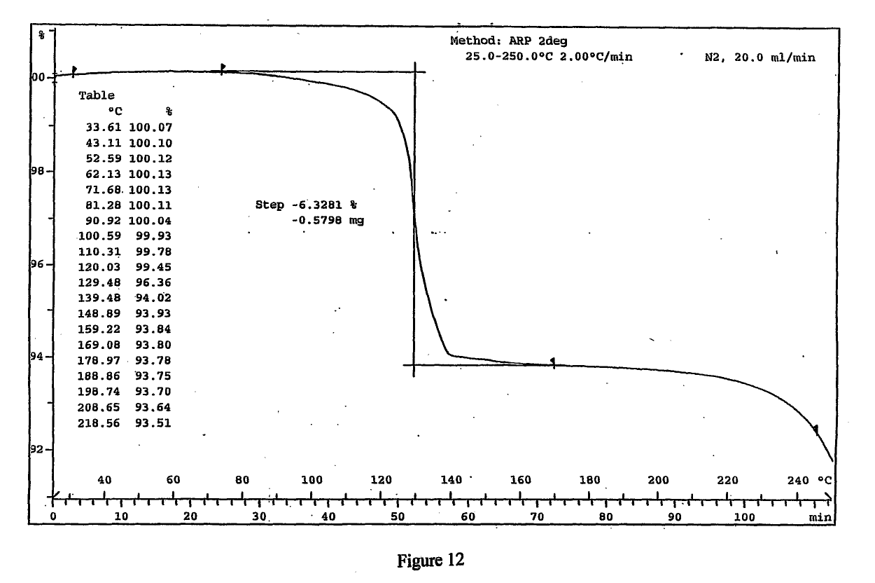

### DETAILED DESCRIPTION OF THE INVENTION

Thus according to the present invention Aripiprazole acid salt on basification (base is selectively alkali hydroxides, such as sodium hydroxide, potassium hydroxide, lithium hydroxide, ammonia, organic bases such as triethylamine, dimethylamine, methylamine, diisopropyl ethyl amine, diisopropylamine, dibutylamine, more preferably triethylamine, dimethylamine) at about 50° C. to about 90° C. in a mixture of water and organic ester solvent, (the organic solvent selected from ethyl acetate, isopropyl acetate), separating the solvent layers, washing the organic layer with water, concentrating the organic layer to reduce the water content to below 0.5%, raising the temperature to about 65° C.-90° C., maintaining at the temperature at about 65° C. to 90° C. for about 10 min to 8 hrs, cooling to about 15° C. to about 40° C., mixing for about 30 min-6 hrs, isolating and further drying at temperature of about 40° C.-90° C. gives the Aripiprazole Form-B.

In another embodiment of the present invention Aripiprazole form-I is prepared from Aripiprazole acid salt by basification of Aripiprazole acid salt with base (base selectively alkali hydroxides, such as sodium hydroxide, potassium hydroxide, lithium hydroxide, alkali carbonates such as sodium carbonate, potassium carbonate, lithium carbonate, barium carbonate, bicarbonates such as sodium bicarbonate, potassium bicarbonate, ammonia, organic bases selected from triethylamine, dimethylamine, methylamine, more preferably triethylamine, dimethylamine) in a mixture of water and water immiscible organic solvent, preferably methylene dichloride for about 10 min to 2 hrs, separating the layers, washing the organic layer with water, removal of the solvent from the organic layer, dissolution of residue in organic polar solvent such as DMF,DMA if required by heating 30° C.-65° C., cooling to low temperature about −15° C. to 20° C. isolating or optionally adding ante solvent (ante solvent selectively ketones such as acetone, methyl ethyl ketone, methyl isobutyl ketone, ethers such as diethyl ether, diisopropyl ether, methyl tert butyl ether, hydrocarbons such as cyclohexane, n-hexane, n-heptane, and esters such as ethyl acetate, isopropyl acetate), at temperature of about 35° C. to followed by cooling to low temperature such as about −5° C. to about 35° C., preferably 5° C. to about 20° C., isolating and drying at temperature of about 35° C. to about 65° C. gives the Aripiprazole form-I.

In another embodiment of the present invention Aripiprazole acetic acid solvate is prepared from Aripiprazole acid salt by basification of Aripiprazole acid salt with base (base selectively alkali hydroxides, such as sodium hydroxide, potassium hydroxide, lithium hydroxide, alkali carbonates such as sodium carbonate, potassium carbonate, lithium carbonate, barium carbonate, bicarbonates such as sodium bicarbonate, potassium bicarbonate, ammonia, organic bases selected from triethylamine, dimethylamine, methylamine, more preferably triethylamine, dimethylamine) at about 50° C.-90° C. in a mixture of water-water immiscible organic solvent selected from ethyl acetate, isopropyl acetate, chloroform, toluene, n-butanol for about 10 min-2 hrs, separating the layers, washing the organic layer with water, concentrating the organic layer, adding acetic acid at about 25° C.-75° C., raising the temperature of the reaction mixture to about 65° C.-90° C., adding ante-solvent which is a hydrocarbon or ether; (hydrocarbon such as cyclohexane, n-hexane, n-heptane, methyl cyclohexane, and ether such as methyl tert butyl ether), maintaining the temperature of 65° C. to 90° C. for about 10 min to 8 hrs, cooling to about 35° C.-75° C., seeding with Aripiprazole acetic acid solvate, followed by further cooling to about 15° C.-40° C., mixing for about 30 min—6 hrs, isolating and drying at temperature of about 40° C.-90° C. gives the Aripiprazole acetic acid solvate.

In another embodiment of the invention Aripiprazole methanol solvate is prepared from Aripiprazole acid salt by basification of Aripiprazole acid salt with base (base selectively alkali hydroxides, such as sodium hydroxide, potassium hydroxide, lithium hydroxide, alkali carbonates such as sodium carbonate, potassium carbonate, lithium carbonate, barium carbonate, bicarbonates such as sodium bicarbonate, potassium bicarbonate, ammonia, organic bases such as triethylamine, dimethylamine, methylamine, more preferably triethylamine, dimethylamine) at about 50° C.-about 90° C. in a mixture of water-water immiscible organic solvent such as ethyl acetate, isopropyl acetate for about 10 min to 2 hrs, separating the layers, washing the organic layer with water, concentrating the organic layer, adding 3 to 6 volumes methanol at about 50° C.-90° C. over about 15 min followed by maintaining the temperature at about 50° C.-90° C. for about 15 min-4 hrs, cooling to about 40° C.-10° C., to give the Aripiprazole methanol solvate. Aripiprazole methanol solvate can be dried and the dry material or the wet cake as such can used for the preparation of various polymorphs; of Aripiprazole. The molar ratio of Aripiprazole: methanol is 1:1, in the Aripiprazole methanol solvate.

In another embodiment of the present invention Aripiprazole methanol solvate and Aripiprazole acetic acid solvate suspensions in selected organic solvents when heated to about 450-90° C., maintaining the temperature at about 45° C.-90° C. for about 30 min to 6 hrs, cooling to about 15° C.-35° C., followed by isolation and drying at temperature of about 50° C.-about 90° C. results in polymorphs of Aripiprazole such as Aripiprazole form-B, form-D, form-A, type-I crystals and form-I.

Solvents such as ethyl acetate, isopropyl acetate in the above process results in Aripiprazole Form-B; solvent such as acetonitrile, THF/n-heptane, ethyl acetate/n-heptane result in Form-D; solvent such as aq. Ethanol and water results in Aripiprazole Form-A and solvent such as ethanol results in Aripiprazole type-I crystals; solvent such as DMF, DMA results Aripiprazole form-I.

In another embodiment of the invention Aripiprazole acetic acid solvate is prepared from Aripiprazole methanol solvate by dissolution of Aripiprazole methanol solvate in organic ester solvent, selected from methyl acetate, isopropyl acetate, adding acetic acid at temperature of 45° C. to 75° C., raising the temperature to 60° C.-90° C., followed by slow addition of ante-solvent selected from hydrocarbon of C₅ to C₇ such as cyclohexane, n-hexane, n-heptane, methyl cyclohexane, or aliphatic ether selected from diisopropyl ether, methyl tertbutyl ether, maintenance at temperature of about 60° C. to about 90° C. for about 10 min to 8 hrs, cooling to about 55° C. to 65° C., seeding with Aripiprazole acetic acid solvate followed by cooling to about 15° C. to 40° C., mixing for about 30 min to 6 hrs, followed by isolation and drying at temperature of about 40° C. to about 90° C. gives the Aripiprazole acetic acid solvate.

In another embodiment of the invention the Aripiprazole methanol solvate is prepared from Aripiprazole acetic acid solvate by raising the temperature of a suspension of Aripiprazole acetic acid solvate in methanol to about 40° C. to 70° C., then maintaining for the temperature for about 30 min to 6 hrs, cooling to about 10° C. to 35° C., isolating and drying at about 30° C. to about 60° C. for about 1 hr to about 18 hrs to give Aripiprazole methanol solvate.

Yet another embodiment of the invention is a process for preparation of Aripiprazole acid salts from 7-Hydroxy-3,4-dihydrocarbostyril. Reaction of 7-Hydroxy-3,4-dihydrocarbostyril (I) with 1,4-dibromobutane (II) is carried out in presence of alkali hydroxide such as sodium hydroxide, potassium hydroxide, phase transfer reagent such as quaternary ammonium salts, preferably tetra butyl ammonium bromide, triethyl benzyl ammonium bromide, in alcohol, (preferable alcohol is isopropyl alcohol, methanol, ethanol, butanol and more preferably isopropyl alcohol) at about 45° C., to 90° C. for about 3 hrs to 8 hrs, removing the insolubles if any, removing the solvent along with excess 1,4-dibromobutane below 125° C., cooling, adding alcohol, mixing at about 10° C.-40° C. preferably at about 15° C. to 30° C. for about 30 min to 8 hrs, isolating the acid salt, washing with hydrocarbon such as n-hexane, n-heptane, cyclohexane, methyl cyclohexane, toluene and drying at temperature of about 35° C. to 75° C., preferably at about 40° C. to 50° C. to give 7-(4-bromobutoxy)-3,4-dihydrocarbostyril (III).

Reaction of 7-(41-bromobutoxy)-3,4-dihydrocarbostyril (III) with 1-(2,3-dichlorophenyl)piperazine (IV) is carried out as follows. 7-(4-bromobutoxy)-3,4-dihydrocarbostyril (III) is added to sodium iodide in a short chain alcohol such as methanol, ethanol, isopropanol, butanol, n-propanol, mixed for about 30 min, and triethylamine and 1-(2,3-dichlorophenyl)piperazine are added and the temperature is maintained at about 50° C. to 75° C. for about 12 hrs to 18 hrs followed by cooling to 15° C. to 40° C. to give crude Aripiprazole. The crude Aripiprazole is dissolved in a water immiscible solvent selected from methylene chloride, ethylene dichloride, chloroform, ethyl acetate, isopropyl acetate more preferably in methylene chloride stirred at about 20° C.-50° C. for about 10 min-2 hrs followed by slow addition of acid to the reaction mass at about 10° C. to 30° C. over 15 min to 2 hrs then mixed at about 10° C.-30° C. for about 1 hr to 8 hrs. The product is isolated and dried at about 35° C. to 75° C. to give Aripiprazole acid salt. The acid used may be an organic acid or inorganic acid. The organic acid is selected from citric acid, p-toluene sulfonic acid, benzene sulfonic acid and salicylic acid; inorganic acid is hydrobromic acid.

The acids may be added as neat solid or in form of solution by dissolving in suitable solvent selected from ethyl acetate, acetone and isopropyl acetate.

Alternately the Aripiprazole acid salts may be prepared from the reaction mass directly without isolating the crude Aripiprazole. 7-(4-bromobutoxy)-3,4-dihydrocarbostyril (III) is added to sodium iodide in an organic polar solvent, such as acetonitrile, THF, mixing for about 10 min to 1 hr at reflux temperature, cooling the reaction mass -20° C. to about 40° C., followed by addition of 1-(2,3-dichlorophenyl)piperazine (IV) and triethylamine, maintaining the reaction mass at about 60° C. to about 80° C. for about 2 hrs to about 6 hrs, followed by removal of solvent under vacuum at temperature below 60° C. The residue is dissolved in mixture of water and water immiscible solvent such as methylene chloride, ethylene dichloride, chloroform, ethyl acetate, isopropyl acetate followed by the separation of layers. The organic layer is washed with water and concentrated followed by slow addition of acid to the reaction mass at about 10° C. to about 30° C. over 15 min to about 2 hrs followed by mixing at about 10° C. to about 30° C. for about 1 hr to about 8 hrs. The precipitated product is isolated and dried at about 35° C. to about 75° C. to give the Aripiprazole acid salt.

The Aripiprazole acid salts prepared are Aripiprazole p-toluene sulfonate monohydrate, Aripiprazole benzene sulfonate, Aripiprazole salicylate, Aripiprazole citrate, and Aripiprazole hydro bromide. The advantage of converting the crude Aripiprazole into Aripiprazole acid—addition salt is removal of bis impurity, (V) 7-(4-[1-(7-oxy-3,4-dihydrocarbostyril)]butoxy)-3,4-dihydro-2(1H)-quinolinone, formed during the reaction of 7-Hydroxy-3,4-dihydro carbostyril (I) with 1,4-dibromobutane (II), resulting in Aripiprazole of 98% purity. It may be noted that the methods of prior art give purity of 80-85%.

Purification of Aripiprazole acid salt is carried out by mixing the Aripiprazole acid salt with methanol at temperature of about 25° C. to about 50° C. for about 15 min-4 hrs followed by cooling and maintaining temperature of about 10° C.-30° C. for about 30 min-6 hrs.

The invention is now illustrated with non-limiting examples.

### Example-I

### Preparation of Aripiprazole Form-B from Aripiprazole p-toluene Sulfonate

Aripiprazole p-toluene sulfonate salt (100 g) is suspended in a mixture of ethyl acetate (2000 ml), water (400 ml) and the temperature is raised to 70° C.-75° C. Triethylamine (25.7 g) is slowly added over 20 min and the temperature is maintained at 70° C.-75° C. for about 30 min. The reaction mass is allowed to settle, the layers are separated and aq. layer is extracted with ethyl acetate (300 ml) at 0.70° C.-75° C. The organic layers are combined and washed with water (2×400 ml) at 70° C.-75° C. The organic layer is concentrated to 900 ml by distillation of ethyl acetate (m/c of the mass is below 0.5%). The reaction mass is maintained at reflux temperature for 15 min. The reaction mass is cooled to 25° C. and maintained at 25° C.-30° C. for 60 min. The solid is filtered, washed the wet cake with ethyl acetate (50 ml) and dried at 40° C.-45° C. till constant weight.

The dry wt of the Aripiprazole form-B is 58.0 g (Yield: 68.8%).

The product is identical with the reported Aripiprazole Form-B by its FTIR, DSC and X-ray diffraction values.

Similarly Aripiprazole form-B can be prepared by using other Aripiprazole acid-addition salts such as benzene sulfonate, citrate, salicylate, hydro bromide and using the solvents such as ethyl acetate, isopropyl acetate.

### Example-II

### Preparation of Aripiprazole Form-B from Aripiprazole p-toluene Sulfonate

Aripiprazole p-toluene sulfonate salt (60 g) is suspended in a mixture of n-butanol (600 ml), water (240 ml) and sodium hydroxide solution (4 g in 10 ml of water) is added. The temperature of the reaction mass is raised to 70° C.-75° C. and maintained at that temperature for about 15 min. Reaction mass is allowed to settle, and the layers are separated, and the aqueous layer is extracted with n-butanol (300 ml). Organic layer is combined, washed with water (240 ml) at 70° C.-75° C. and n-butanol is distilled off under vacuum at temperature below 70° C. till volume of reaction mass is 160 ml. The reaction mass is cooled to 25° C. and maintained at 20° C.-25° C. for 60 min. The solid is filtered, and the wet cake is washed with n-butanol (30 ml) and dried at 40° C.-45° C. till constant weight.

The dry wt of the Aripiprazole form-B is 28.8 g (Yield: 68.3%).

### Example-III

### Preparation of Aripiprazole Form-I from Aripiprazole p-toluene Sulfonate

Aripiprazole p-toluene sulfonate salt (100 g) is suspended in a mixture of methylene dichloride (900 ml), water (400 ml). Triethylamine (25.7 g) is added slowly over 20 min and maintained at 25° C.-35° C. for about 30 min., allowed to settle, and the layers are separated and the aqueous layer is extracted with methylene dichloride (400 ml) at 0.25° C.-30° C. The organic layers are combined, washed with water (2×400 ml) and dried over anhydrous sodium sulphate (20 g). The solvent is removed by distillation of methylene dichloride followed by vacuum. DMF (70 ml) is added and distilled off to get the residue under vacuum at temperature below 45° C. DMF (140 ml) is added to the residue, the temperature is raised to 50° C. to get clear solution and acetone (280 ml) is slowly added at 50-55° C. over 30 min. The total mass is gradually cooled to 35° C. and further cooled to 10° C. The temperature is maintained at 5° C. to 10° C. for 60 min. The solid is filtered, washed with acetone (50 ml) and the wet cake is slurry washed with acetone (140 ml). Dried the wet cake at 40° C.-45° C. to constant weight.

The dry wt of the Aripiprazole form-I is 55 g (Yield: 78.3%).

The XRD shows peaks at 5.4, 10.0, 10.75, 11.6, 12.6, 15.7, 16.3, 18.5, 19.8, 20.4, 21.8, 22.2, 23.3, 24.5, 26.0, 27.1, 28.8, 32.6 and 33.6±0.2° 2 theta

DSC shows an endotherm at 118.47° C. and 148.47° C.

IR shows the absorptions at 3193, 2939, 2830, 2804, 1680, 1628, 1593, 1579, 1520, 1479, 1449, 1375, 1270, 1192, 1169, 965, 949, 869, 780, 712, 672 and 588±2 cm⁻¹

Aripiprazole form-I can be prepared by using other Aripiprazole acid salts, various solvents, and ante-solvents by following the similar procedure as in example-III and the results are given in the table-1

TABLE 1
(Aripiprazole form-I)

| | | Organic | | |
|---|---|---|---|---|
| | | polar | | |
| S. No. | Aripiprazole acid salt | solvent | Ante-solvent | Yield |
| 1 | p-Toluene sulfonate | DMF | — | 40.0% |
| 2 | p-Toluene sulfonate | DMF | Acetone | 78.3% |
| 3 | p-Toluene sulfonate | DMF | Cyclohexane | 42.8% |
| 4 | p-Toluene sulfonate | DMF | Ethyl acetate | 46.2% |
| 5 | p-Toluene sulfonate | DMF | n-Hexane | 40.8% |
| 6 | p-Toluene sulfonate | DMF | Methyl tert butyl | 54.3% |
| | | | ether | |
| 7 | p-Toluene sulfonate | DMF | n-Heptane | 45.7% |
| 8 | p-Toluene sulfonate | DMF | Di isopropyl ether | 51.6% |
| 9 | p-Toluene sulfonate | DMA | Methyl tert butyl | 48.5% |
| | | | ether | |
| 10 | Benzene sulfonate | DMF | Acetone | 68.4% |
| 11 | Citrate | DMF | Cyclohexane | 77.2% |
| 12 | Salicylate | DMF | n-Heptane | 56.2% |
| 13 | Hydro bromide | DMF | Cyclohexane | 81.3% |

### Example-IV

### Preparation of Aripiprazole Acetic Acid Solvate from Aripiprazole p-toluene Sulfonate

Aripiprazole p-toluene sulfonate salt (100 g) is suspended in a mixture of isopropyl acetate (2000 ml), water (400 ml) and raised the temperature to 70° C.-75° C. Triethylamine (25.7 g) is added slowly over 20 min and maintained at 70° C.-75° C. for about 30 min., allowed to settle, and the layers are separated and the aqueous layer is extracted with isopropyl acetate (300 ml) at 70° C.-75° C. The organic layers are combined and washed with water (2×400 ml) at 70° C.-75° C. The reaction mass is concentrated to 900 ml by distillation of isopropyl acetate (m/c of the reaction mass becomes below 0.5%). Acetic acid (25 ml) is added, the temperature is raised to reflux (83° C. to 86° C.) and cyclohexane (900 ml) is slowly added at reflux temperature over 30 min. The reaction mass is maintained at reflux temperature (75° C. to 78° C.) for about 1 hr, then cooled to 63° C., seeded with Aripiprazole acetic acid solvate (500 mg) and further cooled to 35° C. The temperature is maintained at 25° C. to 35° C. for 30 min. The solid is filtered, washed with cyclohexane (50 ml) and dried at 40° C.-50° C. to constant weight.

The dry wt of the Aripiprazole acetic acid solvate is 51 g (Yield: 72.65%).

The XRD shows peaks at 10.1, 17.4, 18.0, 19.7, 23.3, 24.2, 27.8°±0.2° 2 theta

DSC shows an endotherm at 125.7° C.

IR shows the absorptions at 2947, 2901, 1674, 1521, 1381, 1274, 1172, 1048, 856, 781 cm⁻¹.

Aripiprazole acetic acid solvate can be prepared by using other Aripiprazole acid salts, various solvents, and ante-solvents by following the similar procedure as in example-IV and the results are given in the table-2

TABLE 2
(Aripiprazole acetic acid solvate)

| S. No | Aripiprazole acid salt | Organic solvent | Ante-solvent | Yield |
|---|---|---|---|---|
| 1 | p-Toluene sulfonate | Isopropyl | n-Heptane | 72.65% |
| 2 | p-Toluene sulfonate | Ethyl acetate | Cyclohexane | 76.9% |
| 3 | p-Toluene sulfonate | Toluene | Cyclohexane | 54.8% |
| 4 | p-Toluene sulfonate | Chloroform | Cyclohexane | 68.4% |
| 5 | p-Toluene sulfonate | Isopropyl | n-Hexane | 83.6% |
| 6 | p-Toluene sulfonate | Isopropyl | Methyl tert | 77.1% |
| 7 | p-Toluene sulfonate | Ethyl acetate | n-Heptane | 81.5% |
| 8 | p-Toluene sulfonate | Ethyl acetate | n-Hexane | 77.2% |
| 9 | p-Toluene sulfonate | Ethyl acetate | Methyl tert | 75.7% |
| 10 | Benzene sulfonate | Isopropyl | Cyclohexane | 70.34% |
| 11 | Citrate | Isopropyl | Cyclohexane | 81.4% |
| 12 | Salicylate | Isopropyl | Cyclohexane | 49.1% |
| 13 | Hydro bromide | Isopropyl | Cyclohexane | 85.0% |

### Example-V

### Preparation of Aripiprazole Form-A from Aripiprazole p-toluene Sulfonate

Suspend Aripiprazole p-toluene sulfonate salt (60 g) in a mixture of n-butanol (600 ml), water (240 ml) and add sodium hydroxide solution (4 g in 10 ml of water). Raise the temperature of the mass to 70° C.-75° C. and maintain at that temperature for about 15 min. Allow to settle, separate the layers, extract the aqueous layer with n-butanol (300 ml). Combine organic layers, wash with water (240 ml) at 70° C.-75° C. Cool the reaction mass to 10° C. and maintain at 10° C.-12° C. for 60 min. Filter the solid, wash the wet cake with n-butanol (30 ml) and dry at 40° C.-45° C. till constant weight.

The dry wt of the Aripiprazole Form-A is 21 g (Yield: 49.9%).

The product is identical with the reported Aripiprazole Form-A by its FTIR, DSC and X-ray diffraction values.

Similarly Aripiprazole Form-A can be prepared by using other Aripiprazole acid salts, ethyl acetate, isopropyl acetate instead of n-Butanol, without distillation of solvent, by direct cooling following the similar procedure as in example-XV.

### Example-VI

### Preparation of Aripiprazole Form-D from Aripiprazole p-toluene Sulfonate

Aripiprazole p-toluene sulfonate salt (60 g) is suspended in a mixture of n-butanol (600 ml), water (240 ml) and sodium hydroxide solution (4 g in 10 ml of water) is added. The temperature of the mass is raised to 70° C.-75° C. and maintained at that temperature for about 15 min. It is allowed to settle, the layers are separated, the aqueous layer is extracted with n-butanol (300 ml). The organic layer is combined, washed with water (240 ml) at 70° C.-75° C. and n-butanol is distilled off at temperature 75° C.-80° C. under vacuum till the reaction mass volume becomes 200 ml. Slowly cyclohexane (200 ml) is added at temperature 80° C. over 30 min and is maintained at 75° C.-80° C. for 1 hr. The reaction mass is cooled to 30° C. and maintained at 25° C.-30° C. for 30 min. The solid is filtered and the wet cake is washed with cyclohexane (50 ml) and dried at 40° C.-45° C. till constant weight.

The dry wt of the Aripiprazole Form-D is 23.4 g (Yield: 55.6%).

The product is identical with the reported Aripiprazole Form-D by its FTIR, DSC and X-ray diffraction values.

Similarly Aripiprazole Form-D can be prepared by using other Aripiprazole acid salts, using the solvents such as methyl ethyl ketone, THF and cyclohexane, n-hexane, n-heptane as ante-solvent without distillation of first solvent, addition of ante-solvent and cooling.

### Example-VII

### Preparation of Aripiprazole Methanol Solvate from Aripiprazole p-toluene Sulfonate

Aripiprazole p-toluene sulfonate salt (50 g) is suspended in a mixture of isopropyl acetate (1000 ml), water (200 ml) and sodium hydroxide solution (10 g in 10 ml of water) is added. The temperature of the reaction mass is raised to 70° C.-75° C. and maintained for about 30 min. The pH of the reaction mass is adjusted to 11.0 with sodium hydroxide solution. The layers are allowed to settle. The layers are separated and the aqueous layer is extracted with isopropyl acetate (150 ml) at 70° C.-75° C. The organic layers are washed with water (2×200 ml) at 70° C.-75° C. The reaction mass is concentrated to 400 ml by distilling off isopropyl acetate. Methanol (200 ml) is added, and the temperature is raised to reflux and maintained at reflux for about 30 min. The reaction mass is cooled to 35° C., the solid is filtered, washed with methanol (100 ml) and suck dried.

The wt of the Aripiprazole methanol solvate is 36 g (Yield: 95.4%).

HPLC purity: 99.39%, Methanol content: 6.57%

Elemental analysis: C, 59.88%, H, 6.60%, N, 8.62% and calculated values for C₂₁H₃₁Cl₂N₃O₃. C, 59.95%, H, 6.45%, N, 8.74%

IR Spectrum (KBr, cm⁻¹): 3196, 3108, 2948, 2819, 1675, 1628, 1595, 1578, 1522, 1449, 1378, 1335, 1274, 1243, 1197, 1173, 1140, 1127, 1040, 997, 960, 859, 830, 809, 784, 748, 713 and 532.

¹H NMR (300 MHz, CDCl₃, ppm): 1.65-1.85 (m, 4H), 2.49 (t, 2H), 2.51 (t, 2H), 2.59-2.64 (m, 4H), 2.89 (t, 2H), 3.08 (m, 4H), 3.49 (s, 3H), 3.97 (t, 2H), 6.32 (d, 1H), 6.51-6.54 (dd, 1H), 6.94-6.97 (m, 1H), 7.05 (d, 1H), 7.11-7.17 (m, 2H), 8.04 (s, 1H).

¹³C NMR (300 MHz, DMSO-d₆, ppm): 23.19, 24.38, 27.14, 30.90, 50.17, 51.08, 53.12, 58.07, 67.7, 102.2, 108.7, 115.5, 118.5, 124.39, 127.31, 127.34, 128.4, 133.8, 138.1, 151.1, 158.5 and 172.41.

Mass Spectrum (M+): 448.2, 285.1, 218.1, 176.0, and 164.1.

The XRD shows the peaks at 9.4, 10.7, 11.4, 11.8, 12.3, 13.3, 17.3, 18.4, 19.8, 23.3, 24.3, 25.6, 26.8, 28.0, 28.9, 31.2°±0.2 2 theta values

DSC shows endotherm (peaks) at 113, 139° C.

Aripiprazole methanol solvate can be prepared similarly by using other Aripiprazole acid salts and solvents by following the similar procedure as in example-IV and the results are given in the table-3

TABLE 3
(Aripiprazole methanol solvate)

| S. No | Aripiprazole acid salt | Organic solvent | Second solvent | Yield |
|---|---|---|---|---|
| 1 | Benzene sulfonate | Isopropyl | Methanol | 92.3% |
| 2 | Citrate | Isopropyl | Methanol | 90.7% |
| 3 | Salicylate | Isopropyl | Methanol | 94.1% |
| 4 | Hydro bromide | Isopropyl | Methanol | 89.7% |

### Example-VII

### Preparation of Aripiprazole Acetic Acid Solvate from Aripiprazole Methanol Solvate

Aripiprazole methanol solvate (50 g) is suspended in isopropyl acetate (600 ml) and acetic acid (7 ml) is added. The temperature is raised to reflux and cyclohexane (600 ml) is slowly added at reflux temperature over 20 min. The mass is maintained at reflux temperature for about 1 hr, cooled to 60° C. and seeded with Aripiprazole acetic acid solvate (200 mg). The reaction mass is cooled to 30° C. and maintained at 25° C.-30° C. for 30 min. Filter, wash the wet cake with cyclohexane (50 ml) and dry at 40° C.-50° C. till constant weight.

The dry wt of Aripiprazole acetic acid solvate is 42 g (Yield 90.0%)

The product is identical with Aripiprazole acetic acid solvate by its IR, DSC and X-ray diffraction pattern.

### Example-IX

### Preparation of Aripiprazole Type-I Crystals from Aripiprazole Methanol Solvate

Aripiprazole methanol solvate (50 g) is suspended in ethanol (600 ml), the temperature of the reaction mass is raised to reflux and maintained at reflux for about 2 hrs. The reaction mass is cooled to 30° C., filtered, washed the wet cake with ethanol (50 ml) and dried at 45° C.-50° C. till constant weight.

The dry weight of Aripiprazole Type-I crystals is 43 g (78.5%)

Similarly Aripiprazole Type-I crystals can be prepared by treating the Aripiprazole acetic acid solvate with ethanol.

### Example-X

### Preparation of Aripiprazole Form-B from Aripiprazole Methanol Solvate

Aripiprazole methanol solvate (50 g) is suspended in Isopropyl acetate (600 ml), the temperature is raised to reflux and maintained at reflux temperature for about 2 hrs. The reaction mass is cooled, filtered, the wet cake is washed with isopropyl acetate (50 ml) and dried at 50° C.-60° C. till becomes constant weight.

The dry wt of Aripiprazole Form-B is 40 g (Yield 85.7%)

Similarly Aripiprazole Form-B can be prepared by treating the Aripiprazole methanol solvate or Aripiprazole acetic acid solvate with isopropyl acetate, ethyl acetate or by directly drying the Aripiprazole methanol solvate at 80° C. for about 12 hrs.

### Example-XI

### Preparation of Aripiprazole Form-D from Aripiprazole Methanol Solvate

Aripiprazole methanol solvate (50 g) is suspended in acetonitrile (600 ml), the temperature is raised to reflux and maintained at reflux for about 2 hrs. The reaction mass is cooled to 25° C., the solid is filtered, the wet cake is washed with acetonitrile (50 ml) and dried at 55° C.-60° C. till becomes constant weight.

The dry weight of Aripiprazole Form-D is 43.0 g (Yield 91.4%)

Similarly Aripiprazole Form-D can be prepared by treating the Aripiprazole methanol solvate or Aripiprazole acetic acid solvate with acetonitrile, THF/n-Heptane, ethyl acetate/n-heptane.

### Example-XII

### Preparation of Aripiprazole Form-A from Aripiprazole Methanol Solvate

Aripiprazole methanol solvate (50 g) is suspended in 30% aqueous ethanol (600 ml), and the temperature is raised to reflux and maintained at reflux for about 2 hrs. The reaction mass is cooled to 25° C., the solid is filtered, the wet cake is washed with aqueous ethanol (50 ml) and dried at 55° C.-60° C. till becomes constant weight.

The dry weight of Aripiprazole Form-A is 38.0 g (Yield 77.8%)

Similarly Aripiprazole Form-A can be prepared by treating the Aripiprazole methanol solvate or Aripiprazole acetic acid solvate with aqueous ethanol, water.

### Example-XIII

### Preparation of Aripiprazole p-toluenesulfonate

### Step-1:

Sodium hydroxide (29.5 g, 0.737 mole) is added to a suspension of 7-Hydroxy carbostyril (100 g, 0.613 mole) in isopropyl alcohol (1850 ml) and mixed at 25° C. to 30° C. for about 30 min. Tetra butyl ammonium bromide is added (5 g, 0.015 mole) followed by 1,4-Dibromo butane (530 g, 2.45 mole), raised to reflux and maintained at reflux temperature 80° C.-85° C. for 3 hrs. The insolubles are filtered in hot condition, and isopropyl alcohol is distilled off from the filtrate under vacuum at temperature up to 110° C.-115° C. The reaction mass is cooled and isopropyl alcohol (300 ml) is added to the reaction mass, maintained at 30° C.-35° C. for 1 hr. The mass is further cooled and maintained at 20° C.-22° C. for 2 hrs, filtered, washed with isopropyl alcohol (50 ml) to give the wet cake of about 250 g. The wet cake (250 g) is suspended in n-hexane (300 ml), raised the temperature to reflux and maintained for about 60 min. The reaction mass is cooled to a temperature of 25° C.-35° C. and maintained for 1 hr. The mass is filtered; washed and dried the wet cake at 40° C. to 50° C. till becomes constant weight.

The dry weight of 7-(4-bromobutoxy)-3,4-dihydrocarbostyril is 135 g (Yield: 73.8%).

### Step-2:

Sodium iodide (63.7 g, 0.424 mole) is suspended in acetonitrile (1275 ml), mixed for about 10 min and 7-(4-bromobutoxy)-3,4-dihydrocarbostyril (100 g, 0.335 mole) is added. The temperature is raised to reflux maintained for 30 min and cooled to 35° C. 1-(2,3-dichloro phenyl)piperazine (81.5 g, 0.352 mole) is added followed by triethylamine (51.5 g, 0.51 mole) at 25° C.-35° C. to the reaction mass. The temperature of the reaction mass is raised to reflux and maintained at reflux temperature 3 hrs. Acetonitrile is distilled off at temperature below 45° C. under reduced pressure; the residual mass is cooled to 35° C. Methylene chloride (1000 ml), water (500 ml) are added, and the total mass is mixed for 15 min, allowed to settle, the layers are separated and the aqueous layer is extracted with methylene chloride (500 ml). The combined organic layer is washed with water (500 ml) and dried the organic layer over anhydrous sodium sulphate (15 g). Methylene chloride is distilled out initially at atmospheric pressure finally under vacuum. Further methylene chloride (1000 ml) is added and mixed for 15 min to get a clear solution. p-toluene sulfonic acid solution in ethyl acetate (56 g, in 400 ml) is added to the clear solution at a temperature of 25° C.-35° C. over 60 min and maintained at 25° C.-35° C. for 2 hrs. The solid is filtered, the wet cake is washed with ethyl acetate (50 ml) and dried at 40° C.-50° C. till becomes constant weight. The dried material is suspended in methanol (650 ml), the temperature of the mass is raise to 40° C.-45° C. and maintained at that temperature for 30 min. The mass is cooled to 25° C.-35° C. and maintained for 60 min. Filtered, washed the wet cake with methanol (65 ml) and dried at 40° C.-50° C. till becomes constant weight.

The dry weight of Aripiprazole p-toluene sulfonate salt is 110.5 g (51.6%).

Purity by HPLC is 98.6%, Water content: 2.86%

Elemental analysis: C, 56.23%, H, 6.13%, N, 6.53%, S, 4.62% and calculated values for C₃₀H₃₅Cl₂N₃O₅S.H₂O C, 56.42%, H, 5.84%, N, 6.58%, S, 5.02%

IR Spectrum (KBr, cm⁻¹): 3488, 3208, 3130, 3069, 3026, 2954, 1661, 1621, 1595, 1520, 1474, 1448, 1395, 1373, 1333, 1312, 1264, 1224, 1189, 1170, 1117, 1092, 1057, 1031, 1009, 966, 950, 865, 836, 824, 817, 786, 764, 682, 565 and 547

¹H NMR (300 MHz, CDCl₃, ppm): 1.75-1.82 (m, 4H), 2.28 (s, 3H), 2.41 (t, 2H), 2.43 (t, 2H), 2.49-2.54 (m, 4H), 2.79 (t, 2H), 2.99-3.64 (t, 2H), 2.99-3.64 (m, 2H), 6.44 (d, 1H), 6.48-6.52 (dd, 1H), 6.48-6.52 (m, 2H), 7.05-7.12 (m, 1H), 7.05-7.12 (m, 2H), 7.20-7.26 (dd, 1H), 7.33-7.48 (m, 2H), 9.41 (s, OH), 10.02 (s, NH).

¹³C NMR (300 MHz, CDCl₃, ppm): 20.32, 20.72, 23.97, 25.76, 30.72, 47.87, 51.31, 55.32, 66.64, 101.7, 107.49, 115.66, 119.86, 125.35, 125.46, 126.08, 128.07, 128.37, 128.60, 132.72, 137.74, 139.20, 145.4, 149.38, 157.67 and 170.26.

Mass Spectrum (M+): 448

### Example-XIV

### Preparation of Aripiprazole p-toluenesulfonate (Alternate Procedure)

### Step-1:

The step-1 is carried out in the same way as given in example-XII.

### Step-2:

Sodium iodide (69.5 g, 0.463 mole) is suspended in methanol (1500 ml), mixed for about 10 min and 7-(4-bromobutoxy)-3,4-dihydrocarbostyril (100 g, 0.335 mole) is charged into the mixture. The reaction mass is maintained at 25° C.-35° C. for 30 min and triethylamine (69 g, 0.68 mole) is added followed by 1-(2,3-dichloro phenyl)piperazine (90.0 g, 0.39 mole) at 25° C.-35° C. to the reaction mass. The temperature of the reaction mass is raised to reflux and maintained at reflux temperature for 15 hrs. The reaction mass is cooled to 25° C.-35° C. and maintained for 30 min. The solid is filtered, and the wet cake is washed with methanol (100 ml). The weight of the wet cake is 120 g. The wet cake is dissolved in methylene chloride (1000 ml) and p-toluene sulfonic acid solution in ethyl acetate (48 g, in 400 ml) is added at temperature of 20° C.-25° C. over 60 min and maintained at 20° C.-25° C. for 3 hrs. The solid is filtered, and the wet cake is washed with mixture of 1:1 methylene chloride, ethyl acetate (100 ml) and dried at 40° C.-50° C. till becomes constant weight. The dried material is suspended in methanol (650 ml), the temperature is raised to 40° C.-45° C. and maintained the mass at that temperature of for 30 min. The mass is cooled and maintained at 25° C.-35° C. for 60 min. The wet cake is filtered, washed with methanol (65 ml) and dried at 40° C.-45° C. till becomes constant weight.

The dry weight of Aripiprazole p-toluene sulfonate-salt is 140 g (Yield 65.44%).

Purity by HPLC is 0.99.1%

### Example-XV

### Preparation of Aripiprazole Benzene Sulfonate

### Step-1:

The step-1 is carried out in the same way as given in example-XII.

### Step-2:

Sodium iodide (69.5 g, 0.463 mole) is suspended in methanol (1500 ml), mixed for about 10 min and 7-(4-bromobutoxy)-3,4-dihydrocarbostyril (100 g, 0.335 mole) is charged. The reaction mass temperature is raised to 25° C.-35° C. for 30 min and triethylamine (69 g, 0.68 mole) is added followed by 1-(2,3-dichloro phenyl)piperazine (90.0 g, 0.39 mole) at 25° C.-35° C. to the reaction mass. The temperature of the reaction mass is raised to reflux and maintained at reflux temperature for 15 hrs; The reaction mass is cooled to 25° C.-35° C. and maintained for 30 min. The solid is filtered, washed the wet cake with methanol (100 ml). The wet cake weight is 120 g. The wet cake is dissolved in methylene chloride (600 ml) and benzene sulfonic acid solution in ethyl acetate (44.6 g, in 400 ml) is added at a temperature of 20° C.-25° C. over 30 min and is maintained at 20° C.-25° C. for 30 min. The methylene chloride is distilled off under vacuum, ethyl acetate (600 ml) is added and maintained at 20° C.-22° C. for 45 min. The solid is filtered, and the wet cake is washed with ethyl acetate (100 ml) and dried at 40° C.-50° C. till becomes constant weight. The dried material is suspended in methanol (650 ml), the temperature of the mass is raised to 40° C.-45° C. and maintained for 30 min. The reaction mass is cooled to 25° C.-30° C. and maintained for 30 min. The wet cake is filtered and washed with methanol (65 ml) and dry at 40° C.-45° C. till becomes constant weight.

The dry weight of Aripiprazole benzene sulfonate salt is 88.4 g (Yield 43.5%).

Purity by HPLC is 98.4%

Elemental analysis: C, 57.48%, H, 5.41%, N, 6.98% and calculated values for C₂₉H₃₃Cl₂N₃O₅S. C: 57.42%, H, 5.48%, N, 6.93%

IR Spectrum (KBr, cm⁻¹): 3446, 3194, 2979, 2901, 2706, 2619, 1672, 1627, 1596, 1580, 1521, 1479, 144, 6, 1422, 1388, 1331, 1319, 1269, 1234, 1191, 1167, 1119, 1068, 1054, 1032, 1015, 996, 957, 943, 851, 807, 784, 767, 727, 713, 698, 613, 566 and 551.

¹H NMR (300 MHz, DMSO-ds, ppm): 1.75-1.91 (m, 4H), 2.41 (t, 2H), 2.79 (t, 2H), 2.99-3.64 (m, 10H), 3.94 (t, 3H), 6.44 (d, 1H), 6.48-6.52 (dd, 1H), 7.06 (d, 1H), 7.20-7.41 (m, 6H), 7.58-7.63 (m, 2H), 9.42 (brs, 1H), 10.02 (s, 1H).

¹³C NMR (300 MHz, DMSO-d₆, ppm): 20.82, 24.48, 26.26, 31.28, 48.38, 48.30, 51.80, 51.81, 55.81, 67.13, 102.26, 108.0, 116.16, 120.38, 125.86, 125.90, 125.95, 126.59, 128.1, 128.15, 128.88, 128.97, 129.13, 133.23, 139.71, 148.62, 149.88, 158.18 and 170.76.

Mass Spectrum (M+): 448

### Example-XVI

### Preparation of Aripiprazole Salicylate

### Step-1:

The step-1 is carried out in the same way as given in example-XII.

### Step-2:

Sodium iodide (69.5 g, 0.463 mole) is suspended in methanol (1500 ml) and 7-(4-bromobutoxy)-3,4-dihydrocarbostyril (100 g, 0.335 mole) is added. The reaction mass is maintained at 25° C.-35° C. for 30 min and triethylamine (69 g, 0.68 mole) is added followed by 1-(2,3-dichloro phenyl)piperazine (90.0 g, 0.39 mole) at 25° C.-35° C. to the reaction mass. The temperature of the reaction mass is raised to reflux and maintained at reflux temperature for 15 hrs. The reaction mass is cooled to 25° C.-35° C. and maintained for 30 min. The solid is cooled and filtered. The wet cake is washed with methanol (100 ml). The wet cake is dissolved in methylene chloride (600 ml) and salicylic acid solution in ethyl acetate (37.0 g, in 400 ml) is added at temperature of 20° C.-25° C. over 20 min and maintained at 20° C.-25° C. for 60 min. The solid is filtered, washed the wet cake with ethyl acetate (120 ml) and dried at 40° C.-50° C. till becomes constant weight. The dried material is suspended in methanol (600 ml), the mass is raise to a temperature of 40° C.-45° C. and maintained for 30 min. The mass is cooled to a temperature of 25° C.-30° C. and maintained for 30 min. The wet cake is filter, washed with methanol (60 ml) and dried at 40° C.-45° C. till becomes constant weight.

The dry weight of Aripiprazole salicylate salt is 112.0 g (Yield 57.0%).

Purity by HPLC is 0.98.24%

Elemental analysis: C, 60.09%, H, 5.55%, N, 7.12% and calculated values for C₃₀H₃₃Cl₂N₃O₅. C, 61.44%, H, 5.67%, N, 7.16%

IR Spectrum (KBr, cm⁻¹): 3436, 3203, 3059, 2953, 2879, 2839, 1675, 1626, 1593, 1577, 1520, 1486, 1453, 1423, 1381, 1291, 1276, 1260, 1193, 1173, 1137, 1087, 1044, 1025, 979, 960, 941, 859, 830, 810, 795, 764, 708, 667, 586 and 564.

¹H NMR (300 MHz, DMSO-d₆, ppm): 1.75 (s, 4H); 2.39 (t, 2H), 2.79 (t, 2H), 2.92-3.17 (m, 10H), 3.99 (t, 2H), 6.45 (d, 1H), 6.48-6.50 (dd, 1H), 6.71-6.78 (m, 2H), 7.04 (d, 1H), 7.15-7.36 (m, 4H), 7.71-7.75 (dd, 1H).

¹³C NMR (300 MHz, DMSO-d₆, ppm): 20.86, 24.00, 26.10, 30.74, 48.70, 48.72, 51.40, 51.45, 55.74, 66.88, 101.74, 107.56, 115.56, 116.18, 117.34, 117.78, 119.65, 124.96, 126.07, 128.34, 128.51, 130.23; 132.71, 132.90, 139.17, 149.98, 157.74, 161.82, 170.26 and 172.62.

Mass Spectrum (M+): 448

### Example-XVII

### Preparation of Aripiprazole Citrate

Aripiprazole citrate salt can be prepared similarly by using the citric acid instead of salicylic acid by following the same procedure described as in example-XV

The dry weight of Aripiprazole citrate salt is 115.7 g (Yield 53.9%)

Purity by HPLC is 98.91%

Elemental analysis: C, 53.80%, H, 5.37%, N, 6.35% and calculated values for C₂₉H₃₅Cl₂N₃O₉. C, 54.38%, H, 5.51%, N, 6.56%

IR Spectrum (KBr, cm⁻¹): 3469, 3211, 3097, 3065, 2969, 2844, 2726, 2623, 1728, 1639, 1589, 1518, 1452, 1403, 1318, 1275, 1261, 1194, 1170, 1096, 1052, 1045, 1030, 1010, 958, 952, 931, 894, 864, 826, 785, 734, 712, 670, 640 and 566.

¹H NMR (300 MHz, DMSO-d₆, ppm): 1.72 (brs, 4H), 2.39-2.88 (m, 16H), 3.09 (brs, 2H), 3.93 (t, 2H), 6.44 (d, 1H), 6.48-6.51 (dd, 1H), 7.04 (d, 1H), 7.17 (t, 1H), 7.33 (d, 2H), 9.99 (s, 1H).

¹³C NMR (300 MHz, DMSO-d₆, ppm): 21.49, 24.03, 26.27, 30.78, 43.35, 43.55, 43.57, 49.40, 49.42, 51.95, 51.97, 56.31, 67.02, 72.03, 101.78, 107.58, 115.58, 119.71, 124.82, 126.09, 128.41, 128.53, 132.71, 139.21, 150.37, 157.83, 170.33, 171.43, 171.45 and 175.90.

Mass Spectrum (M+): 448

### Example-XVIII

### Preparation of Aripiprazole Hydro Bromide Salt

### Step-1:

The step-1 is to be carried out in the same way as given in example-XII.

### Step-2:

Sodium iodide (69.5 g, 0.463 mole) is suspended in methanol (1500 ml), mixed for about 10 min and charged 7-(4-bromobutoxy)-3,4-dihydrocarbostyril (100 g, 0.335 mole): The reaction mass is maintained at 25° C.-35° C. for 30 min and triethylamine (69 g, 0.68 mole) is added followed by 1-(2,3-dichloro phenyl)piperazine (90.0 g, 0.39 mole) at 25° C.-35° C. The temperature of the reaction mass is raised to reflux and maintained at reflux temperature for 15 hrs. Then reaction mass is cooled to 25° C.-35° C. for 30 min. The solid is filtered and the wet cake is washed with methanol (100 ml). The wet cake is dissolved in methylene chloride (600 ml), and aqueous hydrobromic acid (48%, 30 ml) is added at a temperature of 20° C.-25° C. over 20 min and maintained at 20° C.-25° C. for 60 min. The solid is filtered and the wet cake is washed with methylene chloride (100 ml) and dried at 40° C.-50° C. till becomes constant weight. The dry material is suspended in methanol (750 ml), the temperature of the mass is raised to 40° C.-45° C. and maintained for 30 min. The mass is cooled to 25° C.-30° C. and maintained for 30 min. The wet cake filtered and washed with methanol (100 ml) and dry at 40° C.-45° C. till becomes-constant weight.

The dry weight of Aripiprazole hydro bromide salt is 90.8 g (52.7%)

Purity by HPLC is 98.18%

Elemental analysis: C, 51.99%, H, 5.40%, N, 7.66% and calculated values for C₂₃H₂₈BrCl₂N₃O₂. C, 52.19%, H, 5.33%, N, 7.94%

IR Spectrum (KBr, cm⁻¹): 3426, 3191, 3057, 2953, 2651, 2587, 1692, 1626, 1592, 1520, 1483, 1455, 1378, 1333, 1311, 1271′, 1196, 1171, 1133, 1033, 960, 866, 813, 771, 737, 707 and 569.

¹H NMR (300 MHz, DMSO-d₆, ppm): 1.75-1.85 (m, 4H), 2.41 (t, 2H), 2.79 (t, 2H), 3.03-3.65 (m, 10H), 3.95 (t, 2H), 6.44 (d, 1H), 6.49-6.52 (dd, 1H), 7.06 (d, 1H), 7.21-7.41 (m, 3H), 9.56 (brs, 1H), 10.02 (s, 1H).

¹³C NMR (300 MHz, DMSO-d₆, ppm): 20.17, 23.96, 25.85, 30.71, 47.65, 47.67, 51.15, 51.16, 55.22, 66.68, 101.77, 107.48, 115.62, 119.79, 125.27, 126.02, 128.33, 128.60, 132.69, 139.16, 149.38, 157.64 and 170.21.

Mass Spectrum (M+): 448

### Example-XIX

### Preparation of Aripiprazole Methanol Solvate from Aripiprazole Acetic Acid Solvate

Aripiprazole acetic acid solvate (50 g) is suspended in methanol (600 ml), the temperature is raised to reflux and maintained at reflux temperature for about 2 hrs. The reaction mass is cooled, filtered, washed with methanol (50 ml) and dry at 40° C.-50° C. for 6 hrs.

The dry wt of Aripiprazole methanol solvate is 46 g (Yield 84.9%)

Its DSC, IR and XRD identified the product as Aripiprazole methanol solvate

1-79. (canceled)

80. A process for the preparation of Aripiprazole Form-B from an Aripiprazole acid salt, comprising:
basificating an Aripiprazole acid salt with a base in a mixture of water and an organic ester solvent at a temperature of about 50° C. to 90° C.;
separating resultant layers into organic and inorganic layers;
concentrating the organic layer;
raising the temperature of the organic layer to a temperature between 65° C. and 90° C.;
maintaining the temperature between 65° C. and 90° C. for about 10 min to about 8 hrs;
cooling and isolating Aripiprazole Form-B from the organic layer; and
drying of Aripiprazole Form-B at about 40° C. to 90° C.

81. The process as claimed in claim 80, wherein said base is a base selected from alkali hydroxides, triethylamine, dimethylamine, methylamine, diisopropylethylamine, diisopropylamine, and dibutylamine.

82. The process as claimed in claim 80, wherein said organic ester solvent is ethyl acetate, isopropyl acetate or a mixture thereof.

83. A process for the preparation of Aripiprazole Form-1 from an Aripiprazole acid salt, comprising:
neutralizing an Aripiprazole acid salt with a base in a mixture of water and a water immiscible organic solvent;
separating resultant layers and removing the solvent to obtain a residue;
dissolving the residue in an organic polar solvent at a temperature of 35° C. to 65° C. to obtain a solution;
optionally adding an anti-solvent at a temperature of about 35° C. to 65° C. for about 10 minutes to 8 hours;
cooling the solution to about −5° C. to 35° C. and isolating Aripiprazole Form-1; and
drying the Aripiprazole Form-I at about 40° C. to 90° C.;
wherein the Aripiprazole Form-1 is characterized by X-ray diffraction 2 theta values at 10.0, 10.75, 11.6, 12.6, 15.7, 16.3, 18.5, 19.8, 20.4, 21.8, 22.2, 23.3, 24.5, 26.0, 27.1, and 28.8±0.2°.

84. The process as claimed in claim 83, wherein said base is an organic base or an alkali carbonate or bicarbonate.

85. The process as claimed in claim 83, wherein said water immiscible organic solvent is methylene chloride, said organic polar solvent is DMF, DMA, or a mixture thereof, and said anti-solvent is selected from C₅ to C₇-hydrocarbons, aliphatic ethers, ketones, esters, and mixtures thereof.

86. A process for the preparation of Aripiprazole acetic acid solvate from an Aripiprazole acid salt, comprising:
neutralizing the Aripiprazole acid salt with a base in a mixture of water and a water immiscible organic solvent at a temperature of about 50° C. to 90° C.;
separating resultant organic and inorganic layers;
concentrating the organic layer;
adding acetic acid to the organic layer at a temperature of 25° C. to 75° C.;
raising the temperature of the organic layer to a temperature between 65° C. and 90° C.;
adding an anti-solvent and maintaining the temperature at about 65° C. to 90° C. for about 10 minutes to 8 hours;
seeding the organic layer with Aripiprazole acetic acid solvate at about 35° C. to 75° C.;
cooling the organic layer to about 15° C. to 40° C., isolating Aripiprazole acetic acid solvate, and drying the Aripiprazole acetic acid solvate at about 40° C. to 90° C.;
wherein the Aripiprazole acetic acid solvate is characterized by X-ray diffraction 2-theta values at 10.1, 17.4, 18.0, 19.7, 23.3, and 24.2, and 27.80°±0.2°.

87. The process as claimed in claim 86, wherein said base is selected from alkali hydroxides, alkali carbonates and bicarbonates, ammonia, and organic bases.

88. The process as claimed in claim 86, wherein said water immiscible organic solvent is ethyl acetate, isopropyl acetate, chloroform, toluene, n-butanol, or a mixture thereof.

89. The process as claimed in claim 86, wherein said anti-solvent is selected from C₅ to C₇-hydrocarbons, aliphatic ethers, and mixtures thereof.

90. A process for the preparation of Aripiprazole methanol solvate from an Aripiprazole acid salt, comprising:
neutralizing the Aripiprazole acid salt with a base in a mixture of water and a water-immiscible solvent at a temperature between 50° C. and 90° C.;
separating resultant organic and inorganic layers;
concentrating the organic layer;
adding a volume of methanol to the concentrated organic layer at a temperature of about 50° C. to 90° C.;
maintaining the concentrated organic layer mixture at a temperature of 50° C. to 90° C. for about 15 minutes to 4 hours;
cooling the mixture to 10° C. to 40° C.; and
isolating Aripiprazole methanol solvate therefrom,
wherein the Aripiprazole methanol solvate is characterized by X-ray diffraction 2-theta values at 9.4, 10.7, 11.4, 11.8, 12.3, 13.3, 17.3, 18.4, 19.8, 23.3, 24.3, 25.6, 26.8, 28.0, 28.9, and 31.2°±0.2°.

91. The process as claimed in claim 90, wherein said base is an alkali hydroxide, an alkali carbonate or bicarbonate, ammonia, or an organic base.

92. The process as claimed in claim 90, wherein the water-immiscible solvent is ethyl acetate, isopropyl acetate or a mixture thereof.

93. The process as claimed in claim 90, wherein said volume of methanol with respect to Aripiprazole acid salt is about 3 to 6 volumes.

94. A process for the preparation of Aripiprazole form-B from an Aripiprazole solvate, comprising:
dissolving the Aripiprazole solvate in an organic ester solvent;
raising the temperature to about 45° C. to 90° C.;
maintaining the temperature for about 30 min to 6 hrs;
cooling to about 10° C. to 35° C. and isolating Aripiprazole Form-B; and
drying the Aripiprazole Form-B at about 50° C. to 90° C.

95. The process as claimed in claim 94, wherein the Aripiprazole solvate consists of Aripiprazole methanol solvate or Aripiprazole acetic acid solvate.

96. The process as claimed in claim 94, wherein the organic solvent is ethyl acetate, isopropyl acetate, or a mixture thereof.

97. A process for the preparation of Aripiprazole acetic acid solvate from Aripiprazole methanol solvate, comprising:
dissolving Aripiprazole methanol solvate in an organic ester solvent;
adding acetic acid at a temperature of about 45° C. to 75° C. to obtain a resultant mixture;
raising the temperature to 60° C. to 90° C.;
adding an anti-solvent and maintaining the resultant mixture at a temperature of about 60° C. to 90° C. for 10 min to 8 hrs;
seeding the mixture with Aripiprazole acetic acid solvate at about 55° C. to 65° C.;
cooling the mixture to 15° C. to 40° C. and isolating an Aripiprazole acetic acid solvate product; and
drying the Aripiprazole acetic acid solvate at about 40° C. to 90° C.

98. The process as claimed in claim 97, wherein said organic ester solvent is ethyl acetate, isopropyl acetate, or a mixture thereof.

99. The process as claimed in claim 97, wherein said anti-solvent is selected from C₅ to C₇ hydrocarbons, aliphatic ethers, and mixtures thereof.

100. A process for the preparation of Aripiprazole methanol solvate from Aripiprazole acetic acid solvate, comprising:
forming a suspension of Aripiprazole acetic acid solvate in methanol;
raising the temperature of the suspension to about 40° C. to 70° C.;
maintaining the suspension at a temperature of about 40° C. to 70° C. for about 30 minutes to 6 hours;
cooling the suspension to about 10° C. to 35° C.; and
isolating from the suspension and drying an Aripiprazole methanol solvate product at about 30° C. to 60° C. for about 1 to 18 hours.

101. A process for the preparation of an Aripiprazole acid salt, comprising:
reacting 7-(4-bromobutoxy)-3,4-dihydrocarbostyril with 1-(2,3-dichlorophenyl)piperazine in the presence of sodium iodide and triethylamine in an organic polar solvent at about 50° C.-75° C. for about 12 to 18 hours;
removing the solvent or cooling the solvent to a temperature between 15° C. and 40° C. and isolating crude Aripiprazole;
adding water and a water immiscible solvent to the isolated crude Aripiprazole to obtain two layers;
separating the layers to obtain an organic layer;
adding an acid or an acid solution at about 10° C. to 30° C. to the organic layer;
isolating an Aripiprazole acid salt; and
purifying and drying the Aripiprazole acid salt at 35° C. to 75° C.

102. The process as claimed in claim 101, wherein said organic polar solvent is selected from acetonitrile, methanol, ethanol, propanol, butanol, THF, or a mixture thereof.

103. The process as claimed in claim 101, wherein said water immiscible solvent is selected from methylene chloride, ethylene dichloride, chloroform, ethyl acetate, isopropyl acetate, and mixtures thereof.

104. The process as claimed in claim 101, wherein said acid is an organic or an inorganic acid.

105. The process as claimed in claim 104, wherein said organic acid is selected from aryl sulfonic acids, citric acid, and salicylic acid.

106. The process as claimed in claim 104, wherein said acid is hydrobromic acid.

107. The process as claimed in claim 101, wherein said Aripiprazole acid salt is Aripiprazole p-toluene sulfonate, Aripiprazole benzene sulfonate, Aripiprazole citrate, Aripiprazole salicylate, or Aripiprazole hydrobromide.

108. A process for the preparation of 7-(4-bromobutoxy)-3,4-dihydro carbostyril, comprising:
reacting 7-hydroxy-3,4-dihydrocarbostyril with 1,4-dibromobutane in the presence of an alkali hydroxide and a phase transfer catalyst in alcohol at about 45° C. to 90° C. for about 3 to 8 hours to yield a resultant;
removing insolubles from the resultant;
removing solvent and excess 1,4-dibromo butane at 40° C. to 125° C. from the resultant;
adding alcohol to the resultant;
isolating 7-(4-bromobutoxy)-3,4-dihydrocarbostyril from the resultant, washing the 7-(4-bromobutoxy)-3,4-dihydrocarbostyril with a hydrocarbon at about 20° C. to 70° C., and drying the 7-(4-bromobutoxy)-3,4-dihydrocarbostyril at about 35° C. to 75° C.

109. The process as claimed in claim 108, wherein said alkali hydroxide is sodium hydroxide or potassium hydroxide.

110. The process as claimed in claim 108, wherein said phase transfer catalyst is a quaternary ammonium salt.

111. The process as claimed in claim 110, wherein said quaternary ammonium salt is tetra butyl ammonium bromide.

112. The process as claimed in claim 108, wherein said alcohol is selected from methanol, ethanol, propanol, isopropanol, n-butanol, isobutanol, and tert-butanol.

113. The process as claimed in claim 108, wherein said hydrocarbon is selected from n-hexane, n-heptane, cyclohexane, methyl cyclohexane, toluene and mixtures thereof.

114. Crystalline Aripiprazole form-I, characterized by XRD 2-theta values at 5.4, 10.0, 10.75, 11.6, 12.6, 15.7, 16.3, 18.5, 19.8, 20.4, 21.8, 22.2, 23.3, 24.5, 26.0, 27.1, 28.8, 32.6 and 33.6±0.2° or IR absorptions at 3193, 2939, 2830, 2804, 1680, 1628, 1593, 1579, 1520, 1479, 1449, 1375, 1270, 1192, 1169, 965, 949, 869, 780, 712, 672 and 588±2 cm⁻¹.

115. A crystalline compound Aripiprazole methanol solvate.

116. Crystalline Aripiprazole methanol solvate as claimed in claim 115, characterized by XRD 2-theta values at 9.4, 10.7, 11.4, 11.8, 12.3, 13.3, 17.3, 18.4, 19.8, 23.3, 24.3, 25.6, 26.8, 28.0, 28.9, 31.2°±0.2°.

117. Crystalline Aripiprazole p-toluene sulfonate.

118. Crystalline Aripiprazole p-toluene sulfonate as claimed in claim 117, characterized by IR absorptions at 3488, 3208, 3130, 3069, 3026, 2954, 1661, 1621, 1595, 1520, 1474, 1448, 1395, 1373, 1333, 1312, 1264, 1224, 1189, 1170, 1117, 1092, 1057, 1031, 1009, 966, 950, 865, 836, 824, 817, 786, 764, 682, 565 and 547±2 cm⁻¹.

119. Crystalline Aripiprazole benzenesulfonate.

120. Crystalline Aripiprazole benzene sulfonate as claimed in claim 119, characterized by IR absorptions at 3446, 3194, 2979, 2901, 2706, 2619, 1672, 1627, 1596, 1580, 1521, 1479, 1446, 1422, 1388, 1331, 1319, 1269, 1234, 1191, 1167, 1119, 1068, 1054, 1032, 1015, 996, 957, 943, 851, 807, 784, 767, 727, 713, 698, 613, 566, and 551±cm⁻¹.

121. Crystalline Aripiprazole salicylate.

122. Crystalline Aripiprazole salicylate as claimed in claim 121, characterized by IR absorptions at 3436, 3203, 3059, 2953, 2879, 2839, 1675, 1626, 1593, 1577, 1520, 1486, 1453, 1423, 1381, 1291, 1276, 1260, 1193, 1173, 1137, 1087, 1044, 1025, 979, 960, 941, 859, 830, 810, 795, 764, 708, 667, 586, and 564±2 cm⁻¹.

123. Crystalline Aripiprazole citrate.

124. Crystalline Aripiprazole citrate as claimed in claim 123, characterized by IR absorptions at 3469, 3211, 3097, 3065, 2969, 2844, 2726, 2623, 1728, 1639, 1589, 1518, 1452, 1403, 1318, 1275, 1261, 1194, 1170, 1096, 1052, 1045, 1030, 1010, 958, 952, 931, 894, 864, 826, 785, 734, 712, 670, 640, and 566±2 cm⁻¹.
